(19) United States
(12) Patent Application Publication
(10) Pub. No.: US 2023/0390165 A1
(43) Pub. Date: Dec. 7, 2023

(54) POWDERY SOLID COSMETIC

(71) Applicant: SHISEIDO COMPANY, LTD., Tokyo (JP)

(72) Inventors: Motoharu KIMURA, Tokyo (JP); Tomo OSAWA, Tokyo (JP)

(73) Assignee: SHISEIDO COMPANY, LTD., Tokyo (JP)

(21) Appl. No.: 18/245,768

(22) PCT Filed: Oct. 15, 2021
(86) PCT No.: PCT/JP2021/038270

§ 371 Date: Mar. 17, 2023

(30) Foreign Application Priority Data
Oct. 19, 2020 (JP) 2020-175377

(51) Int. Cl. A61K 8/02; A61K 8/26; A61Q 1/12; A61Q 1/10; A61Q 1/08

(52) U.S. Cl. CPC A61K 8/022; A61K 8/26; A61Q 1/12; A61Q 1/10; A61Q 1/08; A61K 2800/30; A61K 2800/614; A61K 2800/61

(57) ABSTRACT

The invention relates to a talc-free powdery solid cosmetic containing a powder component and an oil-based component that is [1] a powdery solid cosmetic which contains mica (A) having an average particle size of 15 μm or less and mica (B) having an average particle size of 10 μm or more as the powder component and in which the difference between the average particle size of the mica (A) and the average particle size of the mica (B) is 5 μm or more or [2] a powdery solid cosmetic containing 30 mass % or more of mica produced by a wet forming method.

### TECHNICAL FIELD

The present invention relates to a talc-free powdery solid cosmetic.

### BACKGROUND ART

Powdery solid cosmetics such as powdery foundation and eye shadow are formed by packing a cosmetic composition in a container and compressing the composition. At this point, for the purpose of maintaining the impact stability during carrying excellent, talc has been blended in the conventional powdery solid cosmetics (Patent Literature 1).

### CITATION LIST

### Patent Literature

Patent Literature 1: Japanese Patent No. 3781994

### SUMMARY OF INVENTION

### Technical Problem

While talc improves the formability and the impact stability of a powdery solid cosmetic because talc is rich in uneven shapes, a powdery solid cosmetic containing talc has room for improvement in terms of the use feeling such as lack of smoothness of the texture and in terms of the finish property such as powdery appearance after application to the skin.

A purpose of the invention is to provide a powdery solid cosmetic having excellent formability, impact stability, use feeling and finish property even without blending talc.

### Solution to Problem

The present inventors have found that a powdery solid cosmetic in which the problem is solved can be obtained when a talc-free powdery solid cosmetic contains mica types having different particle sizes or when a talc-free powdery solid cosmetic contains a specific amount of mica and is produced by a wet forming method.

The invention relates to the following powdery solid cosmetics.
[1] A powdery solid cosmetic containing a powder component and an oil-based component, wherein the powdery solid cosmetic contains mica (A) having an average particle size of 15 μm or less and mica (B) having an average particle size of 10 μm or more as the powder component, the difference between the average particle size of the mica (A) and the average particle size of the mica (B) is 5 μm or more, and the powdery solid cosmetic does not contain talc.
[2] A powdery solid cosmetic containing a powder component and an oil-based component, the powdery solid cosmetic produced by a wet forming method which contains 30 mass % or more of mica as the powder component and which does not contain talc.
[3] The powdery solid cosmetic described in [1], wherein at least one of the mica (A) and the mica (B) is mica with a surface treated with a silicone elastomer or a metallic soap.
[4] The powdery solid cosmetic described in [1] or [3], wherein at least one of the mica (A) and the mica (B) is synthetic fluorphlogopite iron.
[5] The powdery solid cosmetic described in [2], wherein the mica is mica with a surface treated with a silicone elastomer or a metallic soap.
[6] The powdery solid cosmetic described in [2] or [5], wherein the mica is synthetic fluorphlogopite iron.
[7] The powdery solid cosmetic described in any one of [1] to [6] which further contains spherical powder as the powder component.

### Advantageous Effects of Invention

According to the invention, a powdery solid cosmetic having excellent formability, impact stability, use feeling and finish property can be obtained.

### DESCRIPTION OF EMBODIMENTS

### <Powdery Solid Cosmetic 1>

A first embodiment of the powdery solid cosmetic according to the invention (powdery solid cosmetic 1) is explained.

The powdery solid cosmetic 1 of the invention does not contain talc. That is, the talc content is 0%. When talc is not contained, for example, the texture upon use becomes smooth, and the appearance does not become powdery after application to the skin, resulting in the improvement of the use feeling and the finish property.

The powdery solid cosmetic 1 of the invention contains a powder component and an oil-based component and contains mica (A) having an average particle size of 15 μm or less and mica (B) having an average particle size of 10 μm or more as the powder component, and the difference between the average particle size of the mica (A) and the average particle size of the mica (B) is 5 μm or more. Because two types of mica having different average particle sizes are contained, the formability and the impact stability of the powdery solid cosmetic can be made excellent even when talc is not contained.

The average particle size of the mica (A) is preferably 10 μm or less, more preferably 5 μm or less.

The average particle size of the mica (B) is preferably 20 μm or less, more preferably 15 μm or less.

The difference in the average particle size between the mica (A) and the mica (B) is preferably 5 to 10 μm.

In the invention, the average particle size can be measured by the laser diffraction method. The average particle size by the laser diffraction method is the median size on a volumetric basis, is the particle size corresponding to 50% of the cumulative distribution and can be measured, for example with a laser diffraction particle size distribution analyzer.

As the mica, either synthetic mica or natural mica may be used, and a combination of more than one type may also be used. Synthetic mica types include synthetic fluorphlogopite and synthetic fluorphlogopite iron as cosmetic components, and at least one of the mica (A) and the mica (B) is preferably synthetic fluorphlogopite iron in view of the natural finishing. Titanated mica, which is a typical pearl pigment, is not included in the mica in the invention.

At least one of the mica (A) and the mica (B) is preferably mica with a surface treated with a silicone elastomer or a metallic soap. As a result, the smoothness of the use feeling and the impact stability improve. As the silicone elastomer, those described in WO2017/209077 can be preferably used. The metallic soap is magnesium (Mg) stearate or calcium (Ca) stearate.

The mica (A) content of the powdery solid cosmetic 1 is preferably 5 mass % or more, more preferably 10 mass % or more, particularly preferably 15 mass % or more in view of the smoothness of the texture and the natural finishing. The content is preferably 50 mass % or less in view of the impact stability.

The mica (B) content is preferably 45 mass % or less, more preferably 30 mass % or less in view of the impact stability. The content is preferably 5 mass % or more in view of the use feeling (smoothness of the texture).

The ratio (mass ratio) of the mica (A) and the mica (B) is preferably 1:9 to 10:1, more preferably 3:7 to 7:4 in view of the use feeling, the finish property and the impact stability.

The powdery solid cosmetic 1 of the invention preferably further contains spherical powder as the powder component. As a result, the use feeling (smoothness) of the cosmetic improves.

As the spherical powder, spherical powder having an average particle size of 5 to 10 μm is preferable, and the composition is that of silica, nylon, urethane or the like.

The spherical powder content of the powdery solid cosmetic 1 is preferably 5 mass % to 15 mass % in view of the impact stability when the cosmetic is produced by a dry forming method, and the spherical powder content is preferably 7 mass % to 20 mass % in view of the impact stability when the cosmetic is produced by a wet forming method.

The powdery solid cosmetic 1 of the invention preferably further contains fine particle titanium oxide as the powder component. As a result, the impact stability of the formed material further improves. The primary particle size of the fine particle titanium oxide is preferably 0.1 μm or less, further preferably 0.05 μm or less to improve the impact stability.

The powdery solid cosmetic 1 of the invention preferably further contains a metallic soap as the powder component. As a result, the impact stability of the formed material further improves.

The metallic soap is zinc myristate, magnesium myristate or the like to improve the impact stability, and the average particle size is preferably 20 μm or less.

Powder components other than those above are: extender pigments such as sericite, kaolin, magnesium carbonate, calcium carbonate, aluminum silicate, barium silicate, calcium silicate, magnesium silicate, strontium silicate, a metal tungstate, magnesium, silica, zeolite, barium sulfate, sintered calcium sulfate, calcium phosphate, fluorapatite, hydroxyapatite, a metallic soap, boron nitride, nylon powder, polyethylene powder, cellulose powder, silicone powder and zinc oxide; color materials such as carbon black, iron oxide, titanium oxide, zinc oxide, ultramarine, iron blue, chromium oxide, an organic tar colorant and lake; composite pigments such as titanated mica and iron oxide-coated mica; those obtained by subjecting powder thereof to hydrophobization with a silicone compound, a fluorine-modified silicone compound, a fluorine compound, a higher fatty acid, a higher alcohol, a fatty acid ester, a metallic soap, an amino acid, a quaternary ammonium salt, an alkyl phosphate or the like; and the like.

When a composite pigment such as titanated mica and iron oxide-coated mica is blended in the solid powder cosmetic of the invention, the amount thereof is preferably 5 mass % or less.

The method for hydrophobizing powder may be any method which can hydrophobize the powder, and for example, a general surface treatment method such as a gas phase method, a liquid phase method, an autoclave method and a mechanochemical method can be used.

The powdery solid cosmetic 1 of the invention contains an oil-based component. The oil-based component is not limited as long as the effects of the invention are not impaired and is a liquid oil or fat such as silicone oil, avocado oil, camellia oil, macadamia nut oil, corn oil, olive oil, rapeseed oil, sesame oil, persic oil, wheat germ oil, sasanqua camellia oil, castor oil, linseed oil, safflower oil, cottonseed oil, perilla oil, soybean oil, peanut oil, tea seed oil, kaya oil, rice bran oil, Chinese tung oil, Japanese tung oil, jojoba oil, germ oil, triglycerin, glyceryl trioctanoate and glyceryl triisopalmitate, a solid oil or fat such as cacao butter, coconut oil, hydrogenated coconut oil, palm oil, palm kernel oil, rhus succedanea fruit wax kernel oil, hydrogenated oil, rhus succedanea fruit wax and hydrogenated castor oil, a wax such as beeswax, candelilla wax, cotton wax, carnauba wax, bayberry wax, Chinese wax, montan wax, bran wax, kapok wax, sugarcane wax, hexyl laurate, reduced lanolin, jojoba wax, shellac wax and POE cholesterol ether, a hydrocarbon such as liquid paraffin, ozokerite, squalene, pristane, paraffin, ceresin, squalene, Vaseline and microcrystalline wax, a synthetic ester oil such as isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, isocetyl stearate, isocetyl isostearate, cholesteryl 12-hydroxystearylate, ethylene glycol di-2-ethylhexanoate, a dipentaerythritol fatty acid ester, N-alkylglycol monoisostearate, neopentyl glycol dicaprylate, diisostearyl malate, glyceryl di-2-heptylundecanoate, trimethylolpropane tri-2-ethylhexanoate, trimethylolpropane triisostearate, pentanerythritol tetra-2-ethylhexanoate, glyceryl tri-2-ethylhexanoate, trimethylolpropane triisostearate, cetyl 2-ethylhexanoate, 2-ethylhexyl palmitate, glyceryl trimyristate, glyceride tri-2-heptylundecanoate, castor oil fatty acid methyl ester, oleic acid oil, cetostearyl alcohol, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate and triethyl citrate or the like. Two or more kinds thereof may be used in combination.

The oil-based component content of the powdery solid cosmetic 1 is preferably 5 mass % to 15 mass %, more preferably 7 mass % to 13 mass % in view of the use feeling.

The powdery solid cosmetic 1 of the invention may contain a different component in the range which does not impair the effects of the invention in addition to the essential components.

The different components include components which are generally used for cosmetics including medicines such as moisturizing agents, surfactants, thickeners, vitamins and ultraviolet absorbers, fragrance, antioxidants, preservatives and the like.

The moisturizing agents include glycerin, polyoxyethylene polyoxypropylene dimethyl ether, 1,3-butylene glycol, polyethylene glycol, diglycerin, dipropylene glycol, xylitol, trehalose, erythritol, hyaluronic acid, urea and the like, and one kind or a combination of two or more kinds selected therefrom can be used.

The surfactants include hydrophobic nonionic surfactants and hydrophilic nonionic surfactants.

Examples of the hydrophobic nonionic surfactants include sorbitan fatty acid esters such as sorbitan monoisostearate, sorbitan monolaurate, sorbitan monopalmitate, sorbitan monostearate, sorbitan sesquioleate, sorbitan trioleate, diglycerol sorbitan penta-2-ethylhexanoate and diglycerol sorbitan tetra-2-ethylhexanoate, glyceryl polyglyceryl fatty acids such as glyceryl mono-cottonseed oil fatty acid, glyceryl monoerucate, glyceryl sesquioleate, glyceryl monostearate, glyceryl α,α′-oleate pyroglutamate and glyceryl monostearate malate, propylene glycol fatty acid esters such as propylene glycol monostearate, hydrogenated castor oil derivatives, glycerol alkyl ethers and the like.

Examples of the hydrophilic nonionic surfactants include POE sorbitan fatty acid esters such as POE-sorbitan monooleate, POE-sorbitan monostearate, POE-sorbitan monooleate and POE-sorbitan tetraoleate, POE sorbitol fatty acid esters such as POE-sorbitol monolaurate, POE-sorbitol monooleate, POE-sorbitol pentaoleate and POE-sorbitol monostearate, POE glyceryl fatty acid esters such as POE-glyceryl monostearate, POE-glyceryl monoisostearate and POE-glyceryl triisostearate, POE fatty acid esters such as POE monooleate, POE distearate, POE monodioleate and ethylene glycol sistearate, POE alkyl ethers such as POE lauryl ether, POE oleyl ether, POE stearyl ether, POE behenyl ether, POE 2-octyldodecyl ether and POE cholestanol ether, POE alkylphenyl ethers such as POE octylphenyl ether, POE nonylphenyl ether and POE dinonylphenyl ether, Pluaronics such as Bluronic, POE/POP alkyl ethers such as POE/POP cetyl ether, POE/POP 2-decyltetradecyl ether, POE/POP monobutyl ether, POE/POP hydrogenated lanolin and POE/POP glyceryl ether, tetra POE/tetra POP ethylenediamine condensates such as Tetronic, POE castor oil hydrogenated castor oil derivatives such as POE castor oil, POE hydrogenated castor oil, POE hydrogenated castor oil monoisostearate, POE hydrogenated castor oil triisostearate, POE hydrogenated castor oil monopyroglutamate monoisostearate diester and POE hydrogenated castor oil maleate, POE beeswax/lanolin derivatives such as POE sorbitol beeswax, alkanol amides such as coconut oil fatty acid diethanol amides, lauric acid monoethanolamide and fatty acid isopropanolamide, POE propylene glycol fatty acid esters, POE alkylamines, POE fatty acid amides, sucrose fatty acid esters, POE nonylphenyl formaldehyde condensates, alkylethoxydimetyl amine oxides, trioleylphosphoric acid and the like.

Examples of the preservatives include methylparaben, ethylparaben, propylparaben, butylparaben, phenoxyethanol, N-coconut oil fatty acid acyl L-arginine ethyl DL-pyrrolidone carboxylate, sodium debydroacetate, ethylhexyl glycerin, (silver/zinc/ammonium) zeolite and the like.

### <Powdery Solid Cosmetic 2>

A second embodiment of the powdery solid cosmetic according to the invention (powdery solid cosmetic 2) is explained.

The powdery solid cosmetic 2 of the invention does not contain talc. That is, the talc content is 0%. When talc is not contained, for example, the texture upon use becomes smooth, and the appearance does not become powdery after application to the skin, resulting in the improvement of the use feeling and the finish property.

The powdery solid cosmetic 2 of the invention contains a powder component and an oil-based component and contains mica as the powder component in an amount of 30 mass % or more based on the entire powdery solid cosmetic. Because mica is contained, the formability and the impact stability of the powdery solid cosmetic can be made excellent even when talc is not contained.

As the mica, either synthetic mica or natural mica may be used, and a combination of more than one type may also be used. Synthetic mica types include synthetic fluorphlogopite and synthetic fluorphlogopite iron as cosmetic components, and synthetic fluorphlogopite iron is preferable in view of the natural finishing.

The mica is preferably mica with a surface treated with a silicone elastomer or a metallic soap. As a result, the smoothness of the use feeling and the impact stability improve. As the silicone elastomer, those described in WO2017/209077 can be preferably used. The metallic soap is magnesium (Mg) stearate or calcium (Ca) stearate.

The mica content of the powdery solid cosmetic 2 is preferably 40 mass % or more in view of the use feeling and the finish property. The content is preferably 60 mass % or less in view of the impact stability.

The mica in the powdery solid cosmetic 2 has an average particle size of preferably 5 to 20 μm, more preferably 5 to 10 μm in view of the impact stability. Mica types having different average particle sizes may be blended in combination.

The powdery solid cosmetic 2 of the invention preferably further contains spherical powder as the powder component. As a result, the use feeling (smoothness) of the cosmetic improves.

The spherical powder preferably has an average particle size of 5 to 10 μm, and the composition is preferably that of silica, nylon, urethane or the like.

The spherical powder content of the powdery solid cosmetic 2 is preferably 7 mass % to 20 mass %, more preferably 7 mass % to 18 mass % in view of the impact stability.

The powdery solid cosmetic 2 of the invention preferably further contains at least one of fine particle titanium oxide and a metallic soap as the powder component. As a result, the impact stability of the formed material further improves. Preferable embodiments and amounts of the fine particle titanium oxide and the metallic soap are the same as those of the powdery solid cosmetic 1.

Powder components other than those above include those same as the different powder components described for the powdery solid cosmetic 1.

The powdery solid cosmetic 2 of the invention contains an oil-based component. The oil-based component is not limited as long as the effects of the invention are not impaired but is any of those same as the oil-based components described for the powdery solid cosmetic 1.

The oil-based component content of the powdery solid cosmetic 2 is preferably 5 mass % to 15 mass %, more preferably 7 mass % to 13 mass % in view of the use feeling.

The powdery solid cosmetic 2 of the invention may contain a different component in the range which does not impair the effects of the invention in addition to the essential components.

The different components include those same as the different components described for the powdery solid cosmetic 1.

The powdery solid cosmetic 2 of the invention is produced by a wet forming method. As a result, the formability and the impact stability of the powdery solid cosmetic can be made excellent even when talc is not contained. The wet forming method will be described below.

### <Production Method>

The powdery solid cosmetic 1 can be produced by both a “dry forming method” of mixing/forming the powder component and the oil-based component without using a dispersion medium and a “wet forming method” of filling a container with a slurry obtained by wet mixing the powder component and the oil-based component in a dispersion medium, removing the dispersion medium and forming. To improve the formability and the impact stability of the cosmetic, the powdery solid cosmetic 1 is preferably produced by the wet forming method.

The powdery solid cosmetic 2 is produced by the wet forming method to improve the formability/impact stability of the cosmetic.

The dry forming method and the wet forming method are explained below.

### (Dry Forming Method)

For example, after first mixing the powder component with a stirring mixer such as a Henschel mixer, the oil-based component is added and uniformly mixed, and after crushing with a crusher such as a pulverizer, the mixture is packed in a container. The mixture is further formed by dry pressing.

### (Wet Forming Method)

For example, after first mixing the powder component with a stirring mixer such as a Henschel mixer, the oil-based component is added and uniformly mixed, and thus a cosmetic base is prepared. Next, the cosmetic base is mixed with a solvent to obtain a slurry. The slurry is packed in a container. When the slurry does not spread easily in the container or the like during packing, the slurry can be packed evenly by generating slight vibrations to the extent that the filling does not spill. After filling the container or the like, the solvent is removed to solidify the mixture. The solvent is removed by a general method, such as, for example, air drying, warm drying, hot air drying and vacuum suction.

Although the amount of the solvent differs also with the composition of the powder component and the amount of the oil-based component, the amount is preferably an amount resulting in a viscosity at which removal of air from the slurry and packing in a container or the like are easy, and the amount is generally preferably 0.5 to 1.5 times (mass) the amount of the cosmetic base. When the amount of the solvent is too high, drying requires a long time. Moreover, cracking and shrinkage of the content are caused after drying, and the impact resistance decreases.

The solvent is water, an alcohol such as methanol, ethanol and isopropyl alcohol, benzene, toluene, THF, paraffin, silicone or the like, and one kind or a mixture of two or more kinds can be used depending on the characteristics of the powder component and the oil-based component used. Of these, isopropyl alcohol is preferable in view of the use feeling.

In the case of production by the wet forming method, because the oil-based component or the like is sometimes removed with the solvent, the amount during the production should be increased relative to the aimed amount of the final product. The degree of increase in the amount varies with the kinds and the amounts of the blended components and the solvent, the production conditions and the like and is thus appropriately determined, and an increase to 120 to 150 mass % is preferable in many cases. However, when the compatibility with the solvent is high, an increase to around 200 mass % is sometimes required.

### EXAMPLES

The Examples and the like according to the invention are explained below, but the invention is not limited thereto. The amounts in the prescriptions below are all mass %.

The average particle sizes were measured with a laser diffraction particle size distribution analyzer (manufactured by HORIBA, Ltd., type LA-500).

The assessment methods are as follows.

### (Impact Stability)

The produced powdery foundations were put into special compact cases and dropped from a certain height.

Scores were given based on the numbers of dropping until the formed materials cracked.

When the number of dropping until cracking was five or more, the powdery foundation was determined to have excellent impact stability.

### (Use Feeling (Smoothness of Texture))

Ten expert analysts assessed on a 7-point scale. The decimals were rounded up to the nearest whole numbers. When the score was five or more, the powdery foundation was determined to have excellent use feeling.

### (Finish Property (Natural Appearance))

Ten expert analysts assessed on a 7-point scale. The decimals were rounded up to the nearest whole numbers. When the score was five or more, the powdery foundation was determined to have excellent use feeling.

### Comparative Examples 1-1 to 1-6 and Examples 1-1 to 1-12

Powdery foundations were produced according to the prescriptions shown in Table 1 by a dry forming method.

The assessment results are shown in Table 1.

TABLE 1

| | | | Comparative | Comparative | Comparative | Comparative | Comparative |
|---|---|---|---|---|---|---|---|
| | | | Example | Example | Example | Example | Example |
| | | | 1-1 | 1-2 | 1-3 | 1-4 | 1-5 |
| Powder | Extender | Talc (*1) | 50.21 | 30.21 | — | — | — |
| Component | Pigment | Synthetic fluorphlogopite iron | — | 10.00 | — | 50.21 | — |
| | | (particle size of about 5 μm) (*2) | | | | | |
| | | Elastomer-Treated Synthetic | — | — | — | — | — |
| | | fluorphlogopite iron (particle | | | | | |
| | | size of about 5 μm) | | | | | |
| | | Magnesium Stearate-Treated Synthetic | — | — | — | — | 50.21 |
| | | fluorphlogopite (particle size | | | | | |
| | | of about 10 μm) | | | | | |
| | | Synthetic fluorphlogopite | — | 10.00 | 50.21 | — | — |
| | | (particle size of about 10 μm) (*3) | | | | | |
| | | Synthetic fluorphlogopite | — | — | — | — | — |
| | | (particle size of about 20 μm) (*4) | | | | | |
| | | Natural Muscovite (particle size | — | — | — | — | — |
| | | of about 10 μm) (*5) | | | | | |
| | | Boron Nitride (*6) | 10.00 | 10.00 | 10.00 | 10.00 | 10.00 |
| | Excipient | Zinc Myristate Metallic Soap | 1.00 | 1.00 | 1.00 | 1.00 | 1.00 |
| | Titanium Oxide | Fine Particle Titanium Dioxide (*7) | 10.00 | 10.00 | 10.00 | 10.00 | 10.00 |
| | White Pigment | Pigment-Grade Titanium Dioxide (*8) | 7.00 | 7.00 | 7.00 | 7.00 | 7.00 |
| | Color Material | Silicone-Treated Red Iron Oxide (*9) | 0.40 | 0.40 | 0.40 | 0.40 | 0.40 |
| | | Silicone-Treated Yellow Iron Oxide (*10) | 1.24 | 1.24 | 1.24 | 1.24 | 1.24 |
| | | Silicone-Treated Black Iron Oxide (*11) | 0.15 | 0.15 | 0.15 | 0.13 | 0.15 |
| | Spherical | Spherical Urethane Powder (*12) | 6.00 | 6.00 | 6.00 | 6.00 | 6.00 |
| | Powder | Spherical Nylon Powder (*13) | 4.00 | 4.00 | 4.00 | 4.00 | 4.00 |
| Oil-Based | Semi-solid Oil | Semi-solid Silicone Wax Oil | 0.50 | 0.50 | 0.50 | 0.50 | 0.50 |
| Component | Component | | | | | | |
| | Liquid Oil | Dimethicone Oil | 2.50 | 2.50 | 2.50 | 2.50 | 2.50 |
| | Component | Phenyldimethicone Oil | 3.00 | 3.00 | 3.00 | 3.00 | 3.00 |
| | | Sorbitan Sesquiisostearate | 1.00 | 1.00 | 1.00 | 1.00 | 1.00 |

| | | | | | | |
|---|---|---|---|---|---|---|
| Other | Parsol Ultraviolet Absorber | 3.00 | 3.00 | 3.00 | 3.00 | 3.00 |

| | | | | | |
|---|---|---|---|---|---|
| Total | 100.00 | 100.00 | 100.00 | 100.00 | 100.00 |
| Production Method | Dry | Dry | Dry | Dry | Dry |
| Impact Stability | 10 | 8 | 2 | 3 | 4 |
| Use Feeling (Smoothness of Texture) | 3 | 4 | 5 | 6 | 6 |
| Finishing (Natural Appearance) | 3 | 4 | 5 | 6 | 5 |

| | | | Comparative | | | | |
|---|---|---|---|---|---|---|---|
| | | | Example | Example | Example | Example | Example |
| | | | 1-6 | 1-1 | 1-2 | 1-3 | 1-4 |
| Powder | Extender | Talc (*1) | — | — | — | — | — |
| Component | Pigment | Synthetic fluorphlogopite iron | — | 5.21 | 5.21 | 20.21 | 20.21 |
| | | (particle size of about 5 μm) (*2) | | | | | |
| | | Elastomer-Treated Synthetic | 50.21 | — | — | — | — |
| | | fluorphlogopite iron (particle | | | | | |
| | | size of about 5 μm) | | | | | |
| | | Magnesium Stearate-Treated Synthetic | — | — | — | — | — |
| | | fluorphlogopite (particle size | | | | | |
| | | of about 10 μm) | | | | | |
| | | Synthetic fluorphlogopite | — | 45.00 | — | 30.00 | — |
| | | (particle size of about 10 μm) (*3) | | | | | |
| | | Synthetic fluorphlogopite | — | — | 45.00 | — | 30.00 |
| | | (particle size of about 20 μm) (*4) | | | | | |
| | | Natural Muscovite (particle size | — | — | — | — | — |
| | | of about 10 μm) (*5) | | | | | |
| | | Boron Nitride (*6) | 10.00 | 10.00 | 10.00 | 10.00 | 10.00 |
| | Excipient | Zinc Myristate Metallic Soap | 1.00 | 1.00 | 1.00 | 1.00 | 1.00 |
| | Titanium Oxide | Fine Particle Titanium Dioxide (*7) | 10.00 | 10.00 | 10.00 | 10.00 | 10.00 |
| | White Pigment | Pigment-Grade Titanium Dioxide (*8) | 7.00 | 7.00 | 7.00 | 7.00 | 7.00 |
| | Color Material | Silicone-Treated Red Iron Oxide (*9) | 0.40 | 0.40 | 0.40 | 0.40 | 0.40 |
| | | Silicone-Treated Yellow Iron Oxide (*10) | 1.24 | 1.24 | 1.24 | 1.24 | 1.24 |
| | | Silicone-Treated Black Iron Oxide (*11) | 0.15 | 0.15 | 0.15 | 0.15 | 0.15 |
| | Spherical | Spherical Urethane Powder (*12) | 6.00 | 6.00 | 6.00 | 6.00 | 6.00 |
| | Powder | Spherical Nylon Powder (*13) | 4.00 | 4.00 | 4.00 | 4.00 | 4.00 |
| Oil-Based | Semi-solid Oil | Semi-solid Silicone Wax Oil | 0.50 | 0.50 | 0.50 | 0.50 | 0.50 |
| Component | Component | | | | | | |
| | Liquid Oil | Dimethicone Oil | 2.50 | 2.50 | 2.50 | 2.50 | 2.50 |
| | Component | Phenyldimethicone Oil | 3.00 | 3.00 | 3.00 | 3.00 | 3.00 |
| | | Sorbitan Sesquiisostearate | 1.00 | 1.00 | 1.00 | 1.00 | 1.00 |

| | | | | | | |
|---|---|---|---|---|---|---|
| Other | Parsol Ultraviolet Absorber | 3.00 | 3.00 | 3.00 | 3.00 | 3.00 |

| | | | | | |
|---|---|---|---|---|---|
| Total | 100.00 | 100.00 | 100.00 | 100.00 | 100.00 |
| Production Method | Dry | Dry | Dry | Dry | Dry |
| Impact Stability | 4 | 6 | 5 | 6 | 5 |
| Use Feeling (Smoothness of Texture) | 6 | 6 | 6 | 6 | 6 |
| Finishing (Natural Appearance) | 5 | 5 | 5 | 7 | 6 |

| | | | Example | Example | Example | Example |
|---|---|---|---|---|---|---|
| | | | 1-5 | 1-6 | 1-7 | 1-8 |
| Powder | Extender | Talc (*1) | — | — | — | — |
| Component | Pigment | Synthetic fluorphlogopite iron | 30.00 | 30.00 | 45.21 | — |
| | | (particle size of about 5 μm) (*2) | | | | |
| | | Elastomer-Treated Synthetic | — | — | — | — |
| | | fluorphlogopite iron (particle | | | | |
| | | size of about 5 μm) | | | | |
| | | Magnesium Stearate-Treated Synthetic | — | — | — | — |
| | | fluorphlogopite (particle size | | | | |
| | | of about 10 μm) | | | | |
| | | Synthetic fluorphlogopite (particle | 20.21 | — | 5.00 | 20.21 |
| | | size of about 10 μm) (*3) | | | | |
| | | Synthetic fluorphlogopite (particle | — | — | — | 30.00 |
| | | size of about 20 μm) (*4) | | | | |
| | | Natural Muscovite (particle size | — | 20.21 | — | — |
| | | of about 10 μm) (*5) | | | | |
| | | Boron Nitride (*6) | 10.00 | 10.00 | 10.00 | 10.00 |
| | Excipient | Zinc Myristate Metallic Soap | 1.00 | 1.00 | 1.00 | 1.00 |
| | Titanium Oxide | Fine Particle Titanium Dioxide (*7) | 10.00 | 10.00 | 10.00 | 10.00 |
| | White Pigment | Pigment-Grade Titanium Dioxide (*8) | 7.00 | 7.00 | 7.00 | 7.00 |
| | Color Material | Silicone-Treated Red Iron Oxide (*9) | 0.40 | 0.40 | 0.40 | 0.40 |
| | | Silicone-Treated Yellow Iron Oxide (*10) | 1.24 | 1.24 | 1.24 | 1.24 |
| | | Silicone-Treated Black Iron Oxide (*11) | 0.15 | 0.15 | 0.15 | 0.15 |
| | Spherical | Spherical Urethane Powder (*12) | 6.00 | 6.00 | 6.00 | 6.00 |
| | Powder | Spherical Nylon Powder (*13) | 4.00 | 4.00 | 4.00 | 4.00 |
| Oil-Based | Semi-solid Oil | Semi-solid Silicone Wax Oil | 0.50 | 0.50 | 0.50 | 0.50 |
| Component | Component | | | | | |
| | Liquid Oil | Dimethicone Oil | 2.50 | 2.50 | 2.50 | 2.50 |
| | Component | Phenyldimethicone Oil | 3.00 | 3.00 | 3.00 | 3.00 |
| | | Sorbitan Sesquiisostearate | 1.00 | 1.00 | 1.00 | 1.00 |

| | | | | | |
|---|---|---|---|---|---|
| Other | Parsol Ultraviolet Absorber | 3.00 | 3.00 | 3.00 | 3.00 |

| | | | | |
|---|---|---|---|---|
| Total | 100.00 | 100.00 | 100.00 | 100.00 |
| Production Method | Dry | Dry | Dry | Dry |
| Impact Stability | 7 | 6 | 7 | 6 |
| Use Feeling (Smoothness of Texture) | 7 | 5 | 6 | 5 |
| Finishing (Natural Appearance) | 6 | 6 | 7 | 5 |

| | | | Example | Example | Example | Example |
|---|---|---|---|---|---|---|
| | | | 1-9 | 1-10 | 1-11 | 1-12 |
| Powder | Extender | Talc (*1) | — | — | — | — |
| Component | Pigment | Synthetic fluorphlogopite iron | — | — | — | — |
| | | (particle size of about 5 μm) (*2) | | | | |
| | | Elastomer-Treated Synthetic | — | 30.00 | 5.210 | 45.000 |
| | | fluorphlogopite iron (particle | | | | |
| | | size of about 5 μm) | | | | |
| | | Magnesium Stearate-Treated Synthetic | — | 20.21 | 45.000 | 5.210 |
| | | fluorphlogopite (particle size | | | | |
| | | of about 10 μm) | | | | |
| | | Synthetic fluorphlogopite (particle | 30.00 | — | — | — |
| | | size of about 10 μm) (*3) | | | | |
| | | Synthetic fluorphlogopite (particle | 20.21 | — | — | — |
| | | size of about 20 μm) (*4) | | | | |
| | | Natural Muscovite (particle size | — | — | — | — |
| | | of about 10 μm) (*5) | | | | |
| | | Boron Nitride (*6) | 10.00 | 10.00 | 10.00 | 10.00 |
| | Excipient | Zinc Myristate Metallic Soap | 1.00 | 1.00 | 1.00 | 1.00 |
| | Titanium Oxide | Fine Particle Titanium Dioxide (*7) | 10.00 | 10.00 | 10.00 | 10.00 |
| | White Pigment | Pigment-Grade Titanium Dioxide (*8) | 7.00 | 7.00 | 7.00 | 7.00 |
| | Color Material | Silicone-Treated Red Iron Oxide (*9) | 0.40 | 0.40 | 0.40 | 0.40 |
| | | Silicone-Treated Yellow Iron Oxide (*10) | 1.24 | 1.24 | 1.24 | 1.24 |
| | | Silicone-Treated Black Iron Oxide (*11) | 0.15 | 0.13 | 0.15 | 0.15 |
| | Spherical | Spherical Urethane Powder (*12) | 6.00 | 6.00 | 6.00 | 6.00 |
| | Powder | Spherical Nylon Powder (*13) | 4.00 | 4.00 | 4.00 | 4.00 |
| Oil-Based | Semi-solid Oil | Semi-solid Silicone Wax Oil | 0.50 | 0.50 | 0.50 | 0.50 |
| Component | Component | | | | | |
| | Liquid Oil | Dimethicone Oil | 2.50 | 2.50 | 2.50 | 2.50 |
| | Component | Phenyldimethicone Oil | 3.00 | 3.00 | 3.00 | 3.00 |
| | | Sorbitan Sesquiisostearate | 1.00 | 1.00 | 1.00 | 1.00 |

| | | | | | |
|---|---|---|---|---|---|
| Other | Parsol Ultraviolet Absorber | 3.00 | 3.00 | 3.00 | 3.00 |

| | | | | |
|---|---|---|---|---|
| Total | 100.00 | 100.00 | 100.00 | 100.00 |
| Production Method | Dry | Dry | Dry | Dry |
| Impact Stability | 5 | 10 | 10 | 10 |
| Use Feeling (Smoothness of Texture) | 5 | 7 | 7 | 7 |
| Finishing (Natural Appearance) | 5 | 7 | 6 | 7 |

Powdery foundations were produced according to the prescriptions shown in Table 2 by a wet forming method. As the solvent, an alcohol was used.

The assessment results are shown in Table 2 together with Comparative Examples 1-1 to 1-6.

TABLE 2

| | | | Comparative | Comparative | Comparative | Comparative | Comparative |
|---|---|---|---|---|---|---|---|
| | | | Example | Example | Example | Example | Example |
| | | | 1-1 | 1-2 | 1-3 | 1-4 | 1-5 |
| Powder | Extender | Talc (*1) | 50.21 | 30.21 | — | — | — |
| Component | Pigment | Synthetic fluorphlogopite iron | — | 10.00 | — | 50.21 | — |
| | | (particle size of about 5 μm) (*2) | | | | | |
| | | Elastomer-Treated Synthetic | — | — | — | — | — |
| | | fluorphlogopite iron (particle | | | | | |
| | | size of about 5 μm) | | | | | |
| | | Magnesium Stearate-Treated Synthetic | — | — | — | — | 50.21 |
| | | fluorphlogopite (particle size | | | | | |
| | | of about 10 μm) | | | | | |
| | | Synthetic fluorphlogopite (particle | — | 10.00 | 50.21 | — | — |
| | | size of about 10 μm) (*3) | | | | | |
| | | Synthetic fluorphlogopite (particle | — | — | — | — | — |
| | | size of about 20 μm) (*4) | | | | | |
| | | Natural Muscovite (particle | — | — | — | — | — |
| | | size of about 10 μm) (*5) | | | | | |
| | | Boron Nitride (*6) | 10.00 | 10.00 | 10.00 | 10.00 | 10.00 |
| | Excipient | Zinc Myristate Metallic Soap | 1.00 | 1.00 | 1.00 | 1.00 | 1.00 |
| | Titanium Oxide | Fine Particle Titanium Dioxide (*7) | 10.00 | 10.00 | 10.00 | 10.00 | 10.00 |
| | White Pigment | Pigment-Grade Titanium Dioxide (*8) | 7.00 | 7.00 | 7.00 | 7.00 | 7.00 |
| | Color Material | Silicone-Treated Red Iron Oxide (*9) | 0.40 | 0.40 | 0.40 | 0.40 | 0.40 |
| | | Silicone-Treated Yellow Iron Oxide (*10) | 1.24 | 1.24 | 1.24 | 1.24 | 1.24 |
| | | Silicone-Treated Black Iron Oxide (*11) | 0.15 | 0.15 | 0.15 | 0.15 | 0.15 |
| | Spherical | Spherical Urethane Powder (*12) | 6.00 | 6.00 | 6.00 | 6.00 | 6.00 |
| | Powder | Spherical Nylon Powder (*13) | 4.00 | 4.00 | 4.00 | 4.00 | 4.00 |
| | | Spherical Silicone Powder (*14) | — | — | — | — | — |
| Oil-Based | Semi-solid Oil | Semi-solid Silicone Wax Oil | 0.50 | 0.50 | 0.50 | 0.50 | 0.50 |
| Component | Component | | | | | | |
| | Liquid Oil | Dimethicone Oil | 2.50 | 2.50 | 2.50 | 2.50 | 2.50 |
| | Component | Phenyldimethicone Oil | 3.00 | 3.00 | 3.00 | 3.00 | 3.00 |
| | | Sorbitan Sesquiisostearate | 1.00 | 1.00 | 1.00 | 1.00 | 1.00 |

| | | | | | | |
|---|---|---|---|---|---|---|
| Other | Parsol Ultraviolet Absorber | 3.00 | 3.00 | 3.00 | 3.00 | 3.00 |

| | | | | | |
|---|---|---|---|---|---|
| Total | 100.00 | 100.00 | 100.00 | 100.00 | 100.00 |
| Production Method | Dry | Dry | Dry | Dry | Dry |
| Impact Stability | 10 | 8 | 2 | 3 | 4 |
| Use Feeling (Smoothness of Texture) | 3 | 4 | 5 | 6 | 6 |
| Finishing (Natural Appearance) | 3 | 4 | 5 | 6 | 5 |

| | | | Comparative | | | | |
|---|---|---|---|---|---|---|---|
| | | | Example | Example | Example | Example | Example |
| | | | 1-6 | 2-1 | 2-2 | 2-3 | 2-4 |
| Powder | Extender | Talc (*1) | — | — | — | — | — |
| Component | Pigment | Synthetic fluorphlogopite iron | — | — | — | 50.21 | 45.21 |
| | | (particle size of about 5 μm) (*2) | | | | | |
| | | Elastomer-Treated Synthetic | 50.21 | — | — | — | — |
| | | fluorphlogopite iron (particle | | | | | |
| | | size of about 5 μm) | | | | | |
| | | Magnesium Stearate-Treated Synthetic | — | — | — | — | — |
| | | fluorphlogopite (particle size | | | | | |
| | | of about 10 μm) | | | | | |
| | | Synthetic fluorphlogopite (particle | — | 50.21 | 45.21 | — | — |
| | | size of about 10 μm) (*3) | | | | | |
| | | Synthetic fluorphlogopite (particle | — | — | — | — | — |
| | | size of about 20 μm) (*4) | | | | | |
| | | Natural Muscovite (particle | — | — | — | — | — |
| | | size of about 10 μm) (*5) | | | | | |
| | | Boron Nitride (*6) | 10.00 | 10.00 | 10.00 | 10.00 | 10.00 |
| | Excipient | Zinc Myristate Metallic Soap | 1.00 | 1.00 | 1.00 | 1.00 | 1.00 |
| | Titanium Oxide | Fine Particle Titanium Dioxide (*7) | 10.00 | 10.00 | 10.00 | 10.00 | 10.00 |
| | White Pigment | Pigment-Grade Titanium Dioxide (*8) | 7.00 | 7.00 | 7.00 | 7.00 | 7.00 |
| | Color Material | Silicone-Treated Red Iron Oxide (*9) | 0.40 | 0.40 | 0.40 | 0.40 | 0.40 |
| | | Silicone-Treated Yellow Iron Oxide (*10) | 1.24 | 1.24 | 1.24 | 1.24 | 1.24 |
| | | Silicone-Treated Black Iron Oxide (*11) | 0.15 | 0.15 | 0.15 | 0.15 | 0.15 |
| | Spherical | Spherical Urethane Powder (*12) | 6.00 | 6.00 | 6.00 | 6.00 | 6.00 |
| | Powder | Spherical Nylon Powder (*13) | 4.00 | 4.00 | 7.00 | 4.00 | 7.00 |
| | | Spherical Silicone Powder (*14) | — | — | 2.00 | — | 2.00 |
| Oil-Based | Semi-solid Oil | Semi-solid Silicone Wax Oil | 0.50 | 0.50 | 0.50 | 0.50 | 0.50 |
| Component | Component | | | | | | |
| | Liquid Oil | Dimethicone Oil | 2.50 | 2.50 | 2.50 | 2.50 | 2.50 |
| | Component | Phenyldimethicone Oil | 3.00 | 3.00 | 3.00 | 3.00 | 3.00 |
| | | Sorbitan Sesquiisostearate | 1.00 | 1.00 | 1.00 | 1.00 | 1.00 |

| | | | | | | |
|---|---|---|---|---|---|---|
| Other | Parsol Ultraviolet Absorber | 3.00 | 3.00 | 3.00 | 3.00 | 3.00 |

| | | | | | |
|---|---|---|---|---|---|
| Total | 100.00 | 100.00 | 100.00 | 100.00 | 100.00 |
| Production Method | Dry | Wet | Wet | Wet | Wet |
| Impact Stability | 4 | 6 | 5 | 7 | 6 |
| Use Feeling (Smoothness of Texture) | 6 | 6 | 7 | 6 | 7 |
| Finishing (Natural Appearance) | 5 | 5 | 5 | 7 | 7 |

| | | | Example | Example | Example |
|---|---|---|---|---|---|
| | | | 2-5 | 2-6 | 2-7 |
| Powder | Extender | Talc (*1) | — | — | — |
| Component | Pigment | Synthetic fluorphlogopite iron | — | — | 30.00 |
| | | (particle size of about 5 μm) (*2) | | | |
| | | Elastomer-Treated Synthetic | — | 50.21 | — |
| | | fluorphlogopite iron (particle | | | |
| | | size of about 5 μm) | | | |
| | | Magnesium Stearate-Treated Synthetic | 50.21 | — | — |
| | | fluorphlogopite (particle size | | | |
| | | of about 10 μm) | | | |
| | | Synthetic fluorphlogopite (particle | — | — | 20.21 |
| | | size of about 10 μm) (*3) | | | |
| | | Synthetic fluorphlogopite (particle | — | — | — |
| | | size of about 20 μm) (*4) | | | |
| | | Natural Muscovite (particle size | — | — | — |
| | | of about 10 μm) (*5) | | | |
| | | Boron Nitride (*6) | 10.00 | 10.00 | 1.0.00 |
| | Excipient | Zinc Myristate Metallic Soap | 1.00 | 1.00 | 1.00 |
| | Titanium Oxide | Fine Particle Titanium Dioxide (*7) | 10.00 | 10.00 | 10.00 |
| | White Pigment | Pigment-Grade Titanium Dioxide (*8) | 7.00 | 7.00 | 7.00 |
| | Color Material | Silicone-Treated Red Iron Oxide (*9) | 0.40 | 0.40 | 0.40 |
| | | Silicone-Treated Yellow Iron Oxide (*10) | 1.24 | 1.24 | 1.24 |
| | | Silicone-Treated Black Iron Oxide (*11) | 0.15 | 0.15 | 0.15 |
| | Spherical | Spherical Urethane Powder (*12) | 6.00 | 6.00 | 6.00 |
| | Powder | Spherical Nylon Powder (*13) | 4.00 | 4.00 | 4.00 |
| | | Spherical Silicone Powder (*14) | — | — | — |
| Oil-Based | Semi-solid Oil | Semi-solid Silicone Wax Oil | 0.50 | 0.50 | 0.50 |
| Component | Component | | | | |
| | Liquid Oil | Dimethicone Oil | 2.50 | 2.50 | 2.50 |
| | Component | Phenyldimethicone Oil | 3.00 | 3.00 | 3.00 |
| | | Sorbitan Sesquiisostearate | 1.00 | 1.00 | 1.00 |

| | | | | |
|---|---|---|---|---|
| Other | Parsol Ultraviolet Absorber | 3.00 | 3.00 | 3.00 |

| | | | |
|---|---|---|---|
| Total | 100.00 | 100.00 | 100.00 |
| Production Method | Wet | Wet | Wet |
| Impact Stability | 10 | 10 | 10 |
| Use Feeding (Smoothness of Texture) | 6 | 6 | 6 |
| Finishing (Natural Appearance) | 6 | 7 | 7 |

| | | | Example | Example | Example |
|---|---|---|---|---|---|
| | | | 2-8 | 2-9 | 2-10 |
| Powder | Extender | Talc (*1) | — | — | — |
| Component | Pigment | Synthetic fluorphlogopite iron | 25.00 | 30.00 | — |
| | | (particle size of about 5 μm) (*2) | | | |
| | | Elastomer-Treated Synthetic | — | — | 30.00 |
| | | fluorphlogopite iron (particle | | | |
| | | size of about 5 μm) | | | |
| | | Magnesium Stearate-Treated Synthetic | — | — | 20.21 |
| | | fluorphlogopite (particle size | | | |
| | | of about 10 μm) | | | |
| | | Synthetic fluorphlogopite (particle | 20.21 | — | — |
| | | size of about 10 μm) (*3) | | | |
| | | Synthetic fluorphlogopite (particle | — | — | — |
| | | size of about 20 μm) (*4) | | | |
| | | Natural Muscovite (particle size | — | 20.21 | — |
| | | of about 10 μm) (*5) | | | |
| | | Boron Nitride (*6) | 10.00 | 10.00 | 10.00 |
| | Excipient | Zinc Myristate Metallic Soap | 1.00 | 1.00 | 1.00 |
| | Titanium Oxide | Fine Particle Titanium Dioxide (*7) | 10.00 | 10.00 | 10.00 |
| | White Pigment | Pigment-Grade Titanium Dioxide (*8) | 7.00 | 7.00 | 7.00 |
| | Color Material | Silicone-Treated Red Iron Oxide (*9) | 0.40 | 0.40 | 0.40 |
| | | Silicone-Treated Yellow Iron Oxide (*10) | 1.24 | 1.24 | 1.24 |
| | | Silicone-Treated Black Iron Oxide (*11) | 0.15 | 0.15 | 0.15 |
| | Spherical | Spherical Urethane Powder (*12) | 6.00 | 6.00 | 6.00 |
| | Powder | Spherical Nylon Powder (*13) | 7.00 | 4.00 | 4.00 |
| | | Spherical Silicone Powder (*14) | 2.00 | — | — |
| Oil-Based | Semi-solid Oil | Semi-solid Silicone Wax Oil | 0.50 | 0.50 | 0.50 |
| Component | Component | | | | |
| | Liquid Oil | Dimethicone Oil | 2.50 | 2.50 | 2.50 |
| | Component | Phenyldimethicone Oil | 3.00 | 3.00 | 3.00 |
| | | Sorbitan Sesquiisostearate | 1.00 | 1.00 | 1.00 |

| | | | | |
|---|---|---|---|---|
| Other | Parsol Ultraviolet Absorber | 3.00 | 3.00 | 3.00 |

| | | | |
|---|---|---|---|
| Total | 100.00 | 100.00 | 100.00 |
| Production Method | Wet | Wet | Wet |
| Impact Stability | 12 | 9 | 15 |
| Use Feeding (Smoothness of Texture) | 7 | 7 | 7 |
| Finishing (Natural Appearance) | 7 | 6 | 7 |

Annotations in Table 1 and Table 2
(*1) Talc JA-68R manufactured by Asada Milling Co., Ltd.
(*2) PDM-FE manufactured by Topy Industries, Ltd.
(*3) PDM-8W manufactured by Topy Industries, Ltd.
(*4) PDM-20L manufactured by Topy Industries, Ltd.
(*5) Y-1800 manufactured by Yamaguchi Mica Co., Ltd.
(*6) Ronaflair Boroneige SF-12 manufactured by Merck KGaA
(*7) MT-100TV manufactured by Tayca Corporation
(*8) TIPAQUE CR-50 manufactured by Ishihara Sangyo Kaisha, Ltd.
(*9) OTS-2 red iron oxide No. 216P manufactured by Daito Kasei Kogyo Co., Ltd.
(*10) OTS-2 STN-1 manufactured by Daito Kasei Kogyo Co., Ltd.
(*11) OTS-2 BL-100 manufactured by Daito Kasei Kogyo Co., Ltd.
(*12) D-400 manufactured by Toshiki Pigment Co., Ltd.
(*13) Nylon SP-500 manufactured by Toray Industries, Inc.
(*14) KSP-100 manufactured by Shin-Etsu Chemical Co., Ltd.

As shown above, the powdery solid cosmetics of the invention had excellent impact stability and showed excellent results also in the use feeling and the finish property even without containing talc.

From the results in Table 1, powdery solid cosmetics having excellent impact stability, use feeling and finish property were obtained by combining mica types having different particle sizes in the case of the dry production method, from the comparison between the Comparative Examples and the Examples.

Moreover, the results in Table 2 show that that the impact stability can be improved by producing by a wet production method even when one type of mica is blended, from the comparison between Comparative Examples 1-3 to 1-6 and Examples 2-1, 2-3, 2-5 and 2-6.

Furthermore, the results in Table 2 show that the impact stability can be further improved by combining mica types having different particle sizes and producing by a wet production method, from the comparison between Examples 2-1 to 2-6 and Examples 2-7 to 2-10.

Although the invention has been explained in detail referring to specific embodiments, it is obvious to one skilled in the art that various changes and modifications can be added without departing from the spirit and the scope of the invention. The present application is based on a Japanese patent application filed on Oct. 19, 2020 (patent application No. 2020-175377), and the contents thereof are incorporated here by reference.

### INDUSTRIAL APPLICABILITY

The powdery solid cosmetic of the invention can be applied, for example, to cosmetics of various forms containing a large amount of powder, such as powdery foundation, pressed powder, eye shadow, loose powder and blush.

1. A powdery solid cosmetic containing a powder component and an oil-based component,
wherein the powdery solid cosmetic contains mica (A) having an average particle size of 15 μm or less and mica (B) having an average particle size of 10 μm or more as the powder component,
the difference between the average particle size of the mica (A) and the average particle size of the mica (B) is 5 μm or more, and
the powdery solid cosmetic does not contain talc.

2. A powdery solid cosmetic containing a powder component and an oil-based component, the powdery solid cosmetic produced by a wet forming method
which contains 30 mass % or more of mica as the powder component and
which does not contain talc.

3. The powdery solid cosmetic according to claim 1, wherein at least one of the mica (A) and the mica (B) is mica with a surface treated with a silicone elastomer or a metallic soap.

4. The powdery solid cosmetic according to claim 1, wherein at least one of the mica (A) and the mica (B) is synthetic fluorphlogopite iron.

5. The powdery solid cosmetic according to claim 2, wherein the mica is mica with a surface treated with a silicone elastomer or a metallic soap.

6. The powdery solid cosmetic according to claim 2, wherein the mica is synthetic fluorphlogopite iron.

7. The powdery solid cosmetic according to claim 1 which further contains spherical powder as the powder component.
